(19) United States
(12) Patent Application Publication
(10) Pub. No.: US 2022/0088305 A1
(43) Pub. Date: Mar. 24, 2022

(54) INFUSION PUMP WITH PATIENT WEIGHT CHECK

(71) Applicant: ICU Medical, Inc., San Clemente, CA (US)

(72) Inventor: Dirceu Galvao Cavendish, JR., San Clemente, CA (US)

(21) Appl. No.: 17/478,788

(22) Filed: Sep. 17, 2021

Related U.S. Application Data
(60) Provisional application No. 63/081,183, filed on Sep. 21, 2020.

(51) Int. Cl. A61M 5/172; A61M 5/142; A61M 5/168; G16H 20/17; G16H 10/60

(52) U.S. Cl. CPC A61M 5/1723; A61M 5/142; A61M 5/16886; G16H 20/17; A61M 2205/3327; A61M 2005/14208; A61M 2205/505; A61M 2205/583; A61M 2205/52; G16H 10/60

(57) ABSTRACT

An infusion pump is configured to determine a confidence in a patient weight value that is used to determine one or more treatment parameters. The infusion pump includes a display configured to display, a keypad, a processor in communication with the display, and a memory in communication with the processor. The memory stores instructions that when executed by the processor cause the execution of a patient weight check. The patient weight check determines a patient weight value corresponding to a weight of a medical patient to receive an infusion therapy from the infusion pump according to one or more treatment parameters, and a reference weight value corresponding to a weight estimate of the medical patient. The patient weight check determines that a difference between the patient weight value and the reference weight value violates a threshold condition and provides an indication to the user of the violation. The patient weight check receives a confirmation associated with the patient weight value, determines the one or more treatment parameters using the patient weight value, and provides the infusion therapy to the patient according to the one or more treatment parameters.

### CROSS-REFERENCE TO RELATED APPLICATIONS

This application claims the benefit of priority from U.S. Provisional No. 63/081,183, filed Sep. 21, 2020, which is hereby incorporated by reference in its entirety.

### TECHNICAL FIELD

This disclosure relates to the field of infusion pumps, and particularly to techniques for reducing errors as the user enters data to program an infusion pump treatment therapy.

### BACKGROUND

Infusion pumps for infusing one or more fluids into a medical patient are commonplace in modern healthcare environments. A user may program such infusion pump to infuse a particular drug according to various treatment parameters, such as dose, rate, volume, and/or duration of time. The user may also enter the patient's weight as part of the programming procedure. One or more of the treatment parameters may be determined, at least in part, based upon the patient's weight. Therefore, an error in patient weight entry can result in the use of an incorrect treatment parameter for the patient. Such errors can cause grave harm to the patient. Therefore, it would be helpful for the pump to perform a confidence check on the patient weight value entered by the user, and to notify the user of a low confidence determination prior to initiating therapy and to require that the user review and confirm the patient weight value. In addition, to facilitate such confidence checking, it would be useful to receive at least one other reference weight value, such as an estimated weight value, a measured weight value, a stored weight value, and/or a statistical weight value, associated with the patient.

### SUMMARY

Various techniques for providing patient weight checking are described herein. Although many of the examples are described in the context of an infusion pump within a networked hospital environment, the techniques described herein can be applied to other medical devices in addition to infusion pumps, and other environments, including any networked or non-networked environment. The infusion pumps described herein sometimes may be other medical devices (instead of or including an infusion pump), or non-medical devices, or any combination thereof. In various embodiments, a patient weight check of an infusion pump is configured to determine a patient weight value that can be used to determine one or more treatment parameters for the patient. The infusion pump is also configured to determine one or more reference weight values that are used to determine confidence in the patient weight value. If the patient weight value is beyond a threshold difference from the reference weight value or values, the infusion pump may take a confirmatory action. For example, the infusion pump may inform the user of the low confidence, such as by indicating that the user must confirm the user-entered value prior to initiating therapy. Therapy may be provided by the infusion pump only after such user confirmation is received. Such patient weight checking advantageously enables real time feedback to a user during data entry and can prevent an infusion pump from using an incorrect treatment parameter. The features described herein help prevent delivery of incorrect therapies and reduce clinical errors. These and other embodiments are described in greater detail below with reference to FIGS. 1-6.

### BRIEF DESCRIPTION OF THE DRAWINGS

The embodiments described herein are illustrated by way of example, and not by way of limitation, in the figures of the accompanying drawings in which like references indicate similar elements.

FIG. 1A is a schematic diagram of an example network environment including one or more networked infusion pumps in accordance with aspects of this disclosure.

FIG. 1B is a block diagram of another example network environment, including an example clinical environment and an example cloud environment, in accordance with aspects of this disclosure.

FIG. 1C is a block diagram illustrating components of an example cloud environment in accordance with aspects of the present disclosure.

FIG. 2 is a block diagram illustrating components of an example infusion pump in accordance with aspects of the present disclosure.

FIGS. 3A-3B illustrate example display screens of the infusion pump of FIG. 2 showing one implementation of a patient weight check.

FIG. 4 illustrates an example patient weight check method in accordance with aspects of this disclosure.

FIG. 5 illustrates an example mid-arm circumference accessory configured to provide a reference patient weight value for use by the patient weight check.

FIGS. 6A-6C illustrate measurement tape assemblies of the mid-arm circumference accessory of FIG. 5.

### DETAILED DESCRIPTION

### Introduction

An infusion pump for infusing one or more fluids into a medical patient may be programmed by a user to infuse a particular drug according to various treatment parameters, such as dose, rate, volume, and/or duration of time. Certain clinical values may be entered into the infusion pump by the user, retrieved from a database (e.g., an electronic medical record (“EMR”), etc.) over a hospital network, and/or determined by the pump from one or more sensors (e.g., location). These clinical values may be used to determine one or more of the treatment parameters used to deliver a desired infusion therapy to the medical patient. The patient's weight and a desired drug dose are typically entered into the pump by the user when configuring the pump (although in some cases, the patient's weight and/or the dose are retrieved from an external source, such as, for example, from an EMR or other network storage location). The patient's weight is used by the pump to determine one or more of the treatment parameters, such as a drug delivery rate. For example, the drug dose may be specified in terms of micrograms of drug per kilogram of patient weight per unit time (mcg/kg/min). A drug delivery rate (e.g., mL/hr) can be determined using the drug dose (mcg/kg/min), the patient's weight (kg), and user-selected drug concentration (mg/mL) (drug delivery rate=patient weight x dose/drug concentration). Therefore, an error in patient weight entry can directly cause an error in the amount of drug administered to the patient.

Errors in patient weight entry can result from a variety of causes. Perhaps the most common (and most severe) error occurs when the user enters the patient's weight using the incorrect units. For example, a patient may actually weigh 80 lbs, but the user may enter the patient's weight into the infusion pump as 80 kg, which corresponds to 176 lbs (2.2 times the actual weight). Such an error will result in an incorrect drug delivery rate calculation, and a drug delivery rate of 2.2 times the prescribed rate for the patient.

Other sources of patient weight entry error include, but are not limited to: the user pressing the incorrect key on the keypad (e.g., inadvertently pressing a key adjacent to a desired key—sometimes referred to as a “fat fingers” error); incorrectly guessing the patient weight (e.g., when the patient is unconscious in an Emergency Room for emergency treatment); dyslexia, or switching the order of the digits in patient weight; entering the wrong value into the weight data field; storing an inaccurate weight value in a database (such as an EMR) and using such value to determine treatment parameters; accessing an incorrect patient record from the EMR; mixing up values (e.g., entering temperature instead of weight); using the wrong weight type (e.g., using actual weight instead of dry weight); as well as other error sources.

In various embodiments described herein, the infusion pump determines a therapy patient weight value. For example, the therapy patient weight value may be determined as described above. In addition, the infusion pump determines one or more reference patient weight values. The reference patient weight value may be determined from a variety of sources. For example, the reference weight value may be retrieved from the EMR record associated with the medical patient. In some cases, the reference weight value is retrieved from an external device that is configured to measure or to estimate the patient's weight.

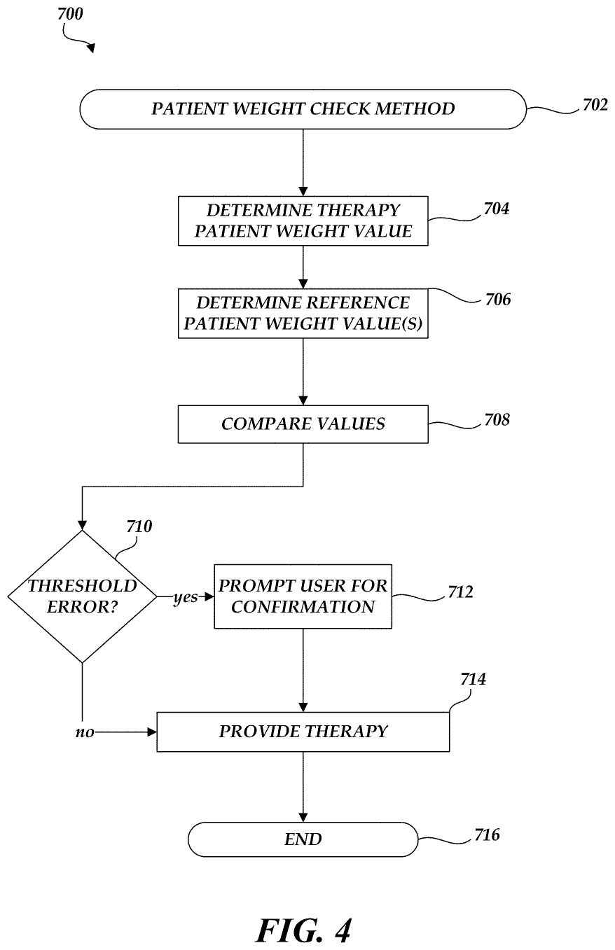

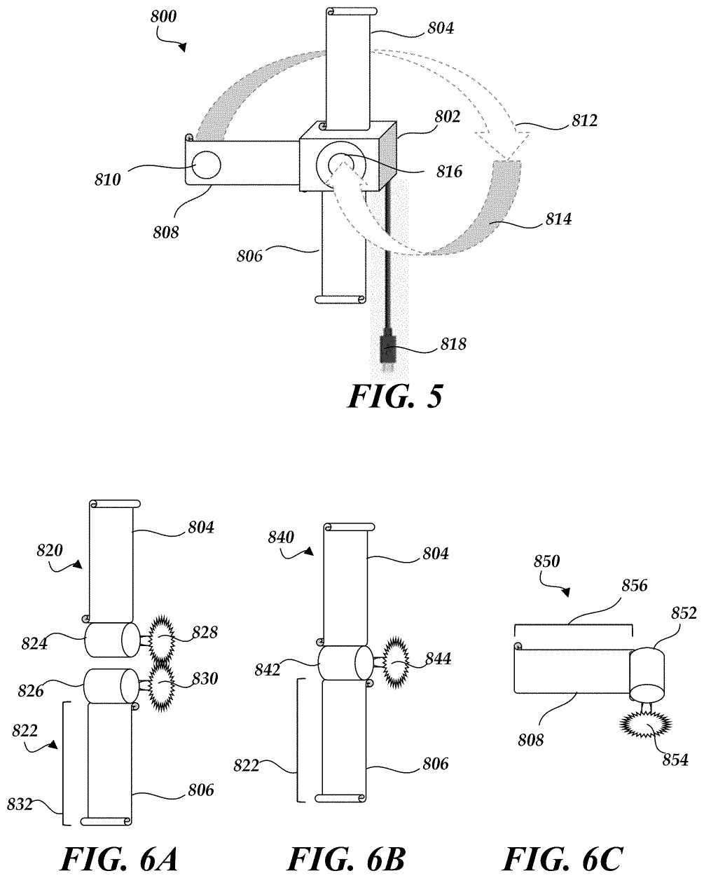

Such external device could include a scale upon which the patient is placed. The scale can include a scale incorporated into a bed or chair configured to measure the patient's weight. In other embodiments, the external device is configured to estimate the patient's weight based upon one or more linear dimensions of the patient's anatomy. Such devices include a mid-arm circumference (“MAC”) weight estimator (examples of which are shown in FIGS. 5-6C), a Mercy Tape (used for pediatric patients between 2 months and 16 years of age), a Broselow-Luten emergency reference tape, etc. The external device may provide a value to the user, and the value may be entered into the pump by the user. However, in some embodiments, the external device is in electrical communication with the infusion pump, for example, via a wired or wireless (BLUETOOTH, near field communication (“NFC”), etc.) communication channel, such that the estimated patient weight value is automatically communicated to the infusion pump without further user data entry. In some embodiments, the reference patient weight value is determined from statistical data, such as the average weight of a patient having one or more of the same age, sex, height, located at the same hospital, located at the same clinical care area within the hospital, etc. as the medical patient.

The therapy patient weight value is compared to the reference patient weight value, and if the difference between the values exceeds a threshold, the user is notified of a potential patient weight entry error, and the user is asked to confirm the entered patient weight value. The infusion pump will not initiate therapy to the patient until a confirmation of the accuracy of the patient weight value is received. The threshold may be configured by an operator (e.g., the user, a technician, a clinician, etc.). For example, the threshold may correspond to a 5%, 10%, 20%, 30%, etc. difference between the therapy patient weight value and the reference patient weight value.

In addition, a drug library accessed by the infusion pump includes one or more rules that define predetermined safe ranges of values for the treatment parameters. For a given drug, the drug library may define lower and upper values of the treatment parameters that have been determined to be safe for use with the particular drug. The lower and upper values may be referred to as “soft limits.” The range of values between the lower and upper soft limits define a safe range of values of the treatment parameter of interest. For a given drug, the drug library may also define lower and upper values of each treatment parameters that may not be exceeded by the user under any circumstance. These lower and upper values may be referred to as “hard limits.” The pump will not allow the selection of a treatment parameter value that is below the lower hard limit or above the upper hard limit under any circumstance. Such rules are generally determined at least based upon the particular drug to be administered by the infusion pump. In addition, the rules for a given drug may vary based upon the concentration of the drug to be administered and/or the clinical care are of the hospital in which the drug is to be administered, and/or other drug library parameters.

The range of values between the soft and hard limits are values that may be selected by the user, but only after confirmation from the user that the user wishes to override the soft limit restriction. The pump keypad may provide the user with an override button for the user to provide such confirmation. Hard limit restrictions may not be overridden.

For example, a user may program a pump to deliver the drug dopamine (of a particular concentration, such as, for example, 400 mg/250 mL) to a medical patient. The pump may access a drug library and determine that the lower and upper hard limits for dosing this particular drug are 0 and 50 mcg/kg/min (micrograms of drug per kilogram of patient weight per minute). Therefore, the user will not be able to enter and submit a dosing value for this drug that is below 0 mcg/kg/min or above 50 mcg/kg/min. The user will not be able to override the hard limit restriction. The drug library may also indicate that the lower and upper soft limits for dosing this particular drug are 2.5 mcg/kg/min and 20 mcg/kg/min. Therefore, the user will not be able to enter and submit a dosing value for this drug that is below 2.5 mcg/kg/min or above 20 mcg/kg/min unless the user activates an override key to override the soft limit restriction. The user may enter and submit a value within the range defined by the lower and upper soft limits (e.g., between 2.5 mcg/kg/min and 20 mcg/kg/min) without restriction.

The user may program other treatment parameters, e.g., infusion rate, volume-to-be-infused (“VTBI”), and/or infusion duration in a similar manner. Selection of such treatment parameter values may be similarly restricted by lower and upper soft and hard limit values for each of such treatment parameters.

The drug library and/or the lower and upper soft and hard limit restrictions may be determined not only based upon the drug to be infused, but also based upon one or more context parameters. Such context parameters include, for example, the drug to be infused, the drug concentration, and/or the clinical care area in which the pump is located. The drug library that is utilized and/or the lower and upper soft and hard limit restrictions may vary based upon the any one or more of the context parameters, such as, for example, location of the infusion pump. If the infusion pump is located in a neonatal intensive care unit (NICU), for example, a drug library may be selected to provide smaller lower and upper soft and limit values for a drug than if the infusion pump were located at location in the hospital that provides treatment to grown adults.

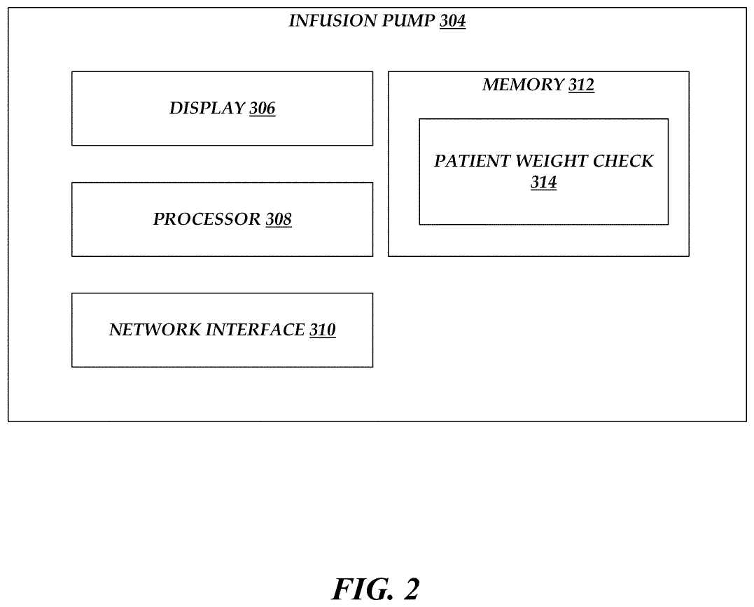

With reference to FIGs. IA-1C, are example network environments in which one or more infusion pumps implementing one of the techniques of the present disclosure may be utilized is described. Following the discussion of FIGs. IA-1C, specific details of the various embodiments of the present disclosure are described with reference to FIGS. 2-6.

Overview of Example Network Environment

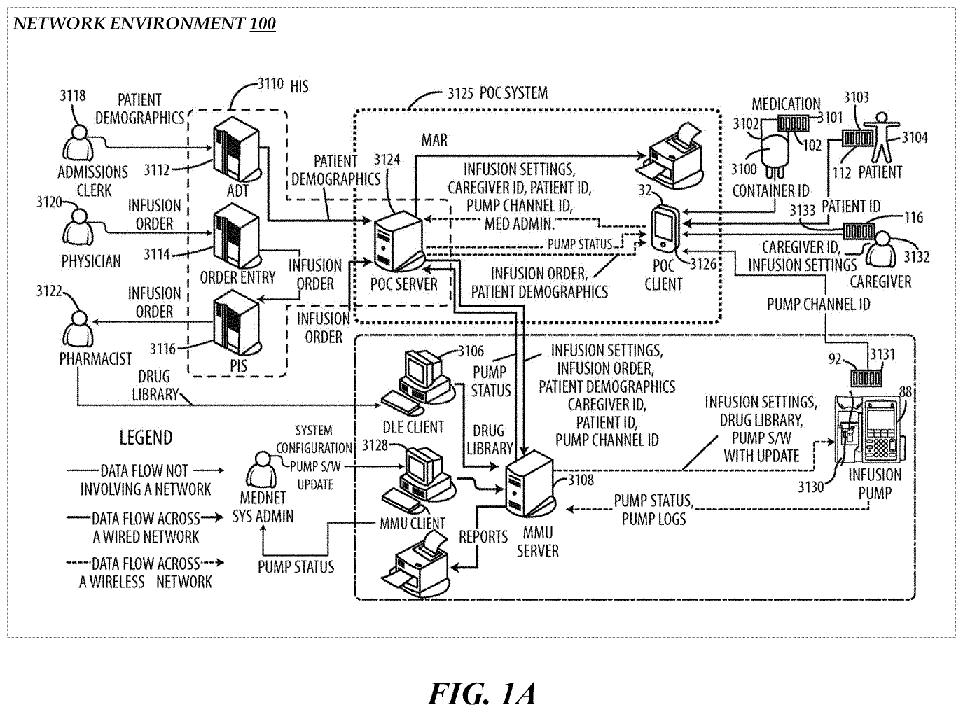

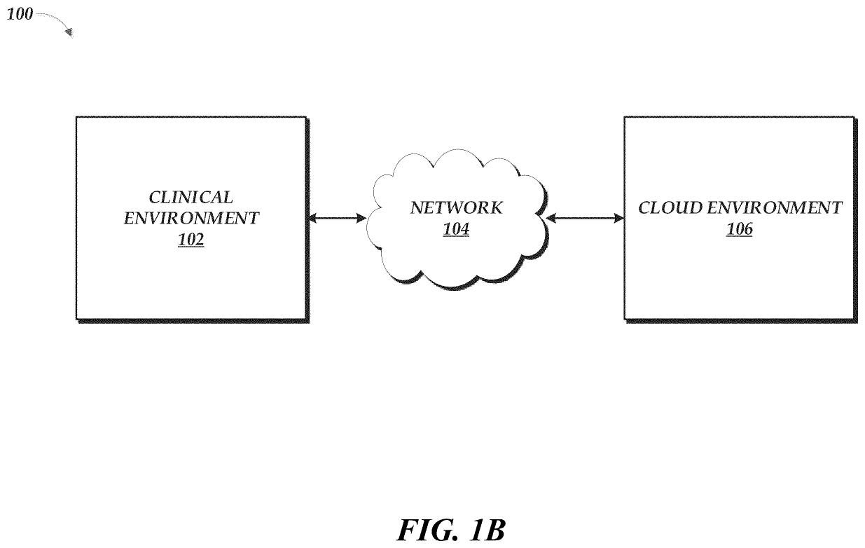

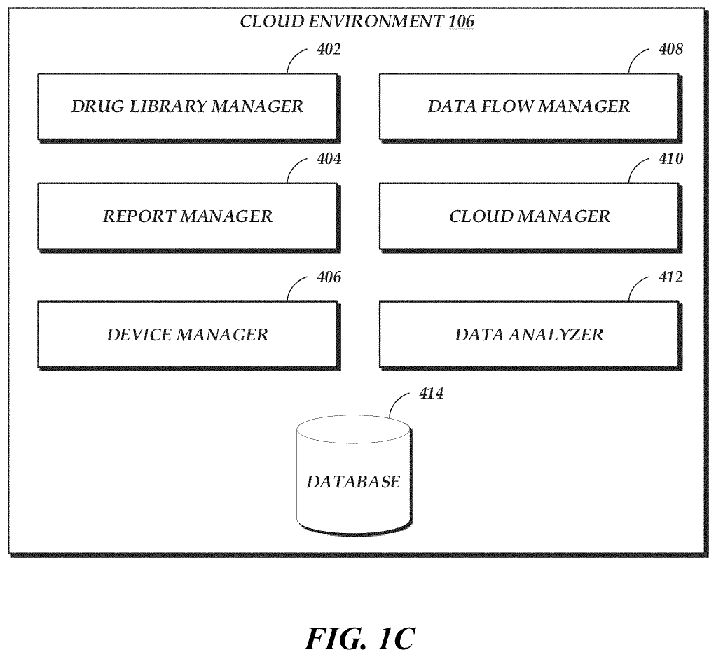

FIG. 1A illustrates one embodiment of a system for administering medication via an infusion pump in a network environment 100. The medication management system (MMS) shown in FIG. lA includes a medication management unit (MMU) server 3108 and a medical device, such as infusion pump 3130, operating in conjunction with one or more information systems or components of a hospital environment. The various components (e.g., systems, servers, computing devices, etc.) of the network environment 100 may be physically located within a single location (e.g., a hospital, clinic, etc.) or may be distributed over multiple locations. For example, the different components of the network environment 100 may communicate with each over the Internet and/or over one or more additional networks. One or more components of the network environment 100 may be located in the cloud, at a remote location from the hospital or clinical environment. For example, in some embodiments, the network environment 100 includes the network embodiment discussed below with respect to FIGS. 1B and 1C.

Intravenous (IV) fluid(s) and/or medication(s) 3100 in containers 3102 may be administered to a patient 3104 using the system shown in FIG. IA. Although the system shown in FIG. lA utilizes barcodes and a barcode reader as apparatus to input and read machine-readable information, those skilled in the art will appreciate that other apparatus for reading or inputting information may be utilized. Moreover, a point of care (POC) client 3126 may include an identification receiver 32 adapted to recognize such indicia may be provided in the MMS.

In certain aspects, the IV fluids and/or medications 3100 in container 3102 may be provided with new or supplemental labels with a unique infusion order identifying barcode by a pharmacist according to certain hospital practices. Specifically, drug container specific identification information, such as barcoded information on the container 3102 may include patient identification information, medication identification information, universal identification information, medical device delivery information, and/or medication order information. The IV fluids and/or medications 3100 in barcode-identified containers 3102 may be supplied to hospitals by various vendors, with preexisting unique barcode identifiers, which include medication information and other information, such as a National Disease Center (NDC) code, expiration information, drug interaction information, and the like.

In some aspects of the disclosure, the universal identification information on the container 3102 may be a unique medication order identifier that, by itself, identifies the order associated with the container. In other aspects, the identification information on the container 3102 may be a composite patient/order code that contains both a patient ID (such as a medical record number) and an order ID unique only within the context of the patient. In certain aspects, the identification information on the container 3102 may include a medication ID. The system identified in FIG. 1A may include a drug library editor (DLE) client 3106, such as a notebook, desktop or server computer. The DLE client 3106 may include DLE software. As described above, the MMU server 3108 may have MMU software that is installed and runs on the MMU server 3108. The drug library and other databases may be stored on the MMU server 3108, on a separate server, and/or in remote locations.

Hospital information systems (HIS) 3110 may include one or more computers connected by cabling, interfaces, and/or Ethernet connections. Alternatively, wireless connections and communications may be used in whole or in part. Servers provide processing capability and memory for storage of data and various application programs or modules, including but not limited to an admissions-discharge-and-transfer (ADT) module or computer 3112, a computerized physician order entry (CPOE) module or computer 3114, and a pharmacy information system (PIS) module or computer 3116. Hospital personnel, such as admission clerks 3118, physicians 3120, and pharmacists 3122, respectively, may be authorized to access these modules through client workstations connected to the servers in order to enter data, access information, run reports, and complete other tasks.

In the embodiment shown in FIG. IA, the HIS 3110 may also include a POC system 3125 including a server or POC computer 3124 (sometimes referred to as a barcode point of care server or computer), or the POC computer 3124 may be separate from the HIS 3110. The POC computer 3124 may act as a part of the POC system 3125 (sometimes referred to as the barcode point of care system or BPOC) and may be able to wirelessly communicate through a plurality of wireless communication nodes located throughout the hospital, utilizing a wireless communications protocol, such as IEEE 801.11, IEEE 802.11, or Bluetooth. The POC computer 3124 may communicate wirelessly with a portable thick client, POC client 3126, carried by a caregiver. The POC client 3126 may be a personal digital assistant (PDA) that includes significant memory, display, and processing capabilities. The POC client device may execute a variety of programs stored in its memory in some degree independently of the POC computer 3124.

In one embodiment of FIG. 1A, the MMU server 3108 may be hard-wired to the DLE client 3106 and to a MMU client 3128. Alternatively, the MMU and DLE client functions may be combined onto a single client computer/workstation or may reside together with the MMU server 3108 on a single combined MMU/DLE server. The MMU server 3108 may reside in a location remote from the patient's room or treatment area. For instance, the MMU server 3108 may reside in a secure, climate-controlled information technology room with other hospital servers, and computer equipment and its client terminals may be located in the pharmacy, biomedical engineering area, nurse station, or ward monitoring area. As discussed above, the MMU server 3108, or any other server or component of the network environment 100, may be located at a remote location, such as in the cloud, or at a server hosted on a remote computing device. One MMU server 3108 may monitor, coordinate, and communicate with many infusion pumps 3130. For example, in one embodiment, the MMU software running on the MMU server 3108 may support up to one thousand infusion pumps concurrently.

In embodiment of FIG. 1A, the POC client 3126 in the POC system 3125 may communicate through the POC server 3124 with the MMU server 3108. The MMU server 3108 may interface or communicate wireles sly with the infusion pump 3130 through the same wireless nodes utilized by the POC system 3125 and a connectivity engine and antenna on or in the infusion pump 3130. Communication between the infusion pump 3130 and the POC client 3126 may take place through the MMU server 3108 and POC server 3124. The MMU server 3108 may store in an associated memory both the logical ID and the network ID or Internet Protocol (IP) address of the infusion pump(s) 3130, such that only the MMU server 3108 may communicate in a direct wireless manner with the infusion pump 3130. Alternatively, the MMU server 3108 may provide the IP address and other information about the infusion pump 3130 to the POC system 3125 to facilitate direct communication between the POC system 3125 and the infusion pump 3130.

Upon admission to the hospital, the admission clerk 3118 or similar personnel may enter demographic information about each patient 3104 into an associated memory of the ADT module or computer 3112 of an HIS database stored in an associated memory of the HIS 3110. Each patient 3104 may be issued a patient identification wristband, bracelet, or tag 112 that may include an identifier 3103, such as a barcode or RFID tag, identifying the patient. The wristband, bracelet, or tag 112 may also include other information, in machine readable or human-readable form, such as the name of the patient's doctor, blood type, allergies, and the like.

The patient's doctor 3120 may prescribe medical treatment by entering a medication order into the CPOE module or computer 3114 within the HIS 3110. The medication order may specify a start time, stop time, a range of allowable doses, physiological targets, route, and site of administration. In the case of an order for infusion of fluids or medication, the order may be written in various formats, and may include the patient's name, patient ID number, a unique medication order or prescription number, a medication name, medication concentration, a dose or dosage, frequency, and/or a time of desired delivery. This information may be entered into the memory of the CPOE module or computer 3114 and may be stored in a memory associated with at least the POC server 3124.

The medication order may also be delivered electronically to the PIS module or computer 3116 in the pharmacy and may be stored in an associated memory. The pharmacist 3122 may screen the prescribed order, translate it into an order for dispensing medication, and prepare the medication or fluids with the proper additives and/or necessary diluents. The pharmacist 3122 may prepare and affix a label 102 with drug container specific identifying information 3101 to the medication or drug container 3102. The label may include in machine-readable and/or human-readable form medical device specific delivery information including but not limited to the dispense ID number, patient ID, drug name, drug concentration, container volume, volume-to-be-infused (“VTBI”), rate, duration, and the like. Only two of the three variables VTBI, rate, and duration may be defined as the third may be calculated when the other two are known. The labeled medication may be delivered to a secure, designated staging location or mobile drug cart on the ward or floor near the patient's room or treatment area. The medication order pending dispensing or administration may be posted to a task list in the HIS 3110 and POC system 3125 and stored in an associated memory.

The caregiver 3132 (e.g., a nurse) may use the identification receiver 32 associated with the POC client 3126 to scan his/her caregiver identification badge 116 and enter a password, which logs the caregiver into the system and authorizes the caregiver to access a nurse's task list from the POC system 3125 through the POC client 3126. The caregiver 3132 may view from the task list that IV drugs are to be administered to certain patients 3104 in certain rooms. The caregiver 3132 obtains the necessary supplies, including medications, from the pharmacy and/or a staging area in the vicinity of the patient's room.

The caregiver 3132 may take the supplies to a patient's bedside, turn on the infusion pump 3130, verify that the network connection icon on the infusion pump 3130 indicates a network connection (for example, a wireless connection such as Wi-Fi or the like) is present, select the appropriate clinical care area (CCA) on the infusion pump 3130, and mount the IV bag, container, or vial 3102 and any associated tube set as required in position relative to the patient 3104 and infusion pump 3130 for infusion. Another connection icon on the infusion pump 3130 or pump user interface screen can indicate that a wired or wireless connection to the MMU server 3108 is present. Using the identification receiver/reader integral to the POC client 3126, the caregiver 3132 may scan the barcode on the patient's identification wristband, bracelet, or tag 112 or other patient identification device. A task list associated with that particular patient may appear on the POC client 3126 screen. The task list, which may also include orders to give other forms of treatment or medication by other routes (oral, topical, etc.), may be obtained from the HIS 3110 via the POC server 3124 and communicated wirelessly to the POC client 3126. In one embodiment, the list is generated by matching the scanned patient ID with the patient ID for orders in memory within the POC server 3124. In another embodiment, the order information may be obtained by scanning the drug container specific identification information for associated orders in memory within the POC server 3124, through the following step(s).

The caregiver 3132 may scan the medication barcode label 102 containing medication container specific identification information 3101 on the medication container 3102 with the POC client 3126. The POC client 3126 may highlight the IV administration task on the task list and send the scanned medication container specific identification information, such as dispense ID information, from the medication container 3102, to the POC server 3124. The POC server may use the medication container specific identification information to pull together the rest of the order details and send them back to the POC client 3126. The POC client 3126 may then display an IV Documentation Form on its screen. One side of the IV Documentation Form screen may show the order details as “ordered” and the other side may be reserved for a status report from the infusion pump 3130. The status report from the infusion pump 3130 may be transmitted to the POC client 3126 through the POC server 3124 and MMU server 3108. The lower portion of the IV Documentation Form screen may provide the caregiver 3132 with instructions (like to scan the infusion pump 3130 barcode) or identify whether the pump is running or stopped.

The caregiver 3132 may then scan the barcode label 92 associated with the infusion pump 3130 (or pump channel if the pump is a multi-channel pump). The barcode label 92 may contain medical device specific identification information 3131, such as the logical name and/or logical address of the device or channel. The POC system 3125 then automatically bundles the information into a program pump request containing the “order details” and in one embodiment, without further interaction with the caregiver 3132, transmits this information to the MMU server 3108.

The program pump request may include at least some of the following information (in HIS/POC system format): a Transaction ID, which may include a Logical Pump ID, a Pump Compartment, a Pump Channel ID, a Reference Device Address, a Caregiver ID, a Caregiver Name, a Patient/Person ID (HIS identifier), a Patient Name, a Patient Birth Date & Time, a Patient Gender, a Patient Weight, a Patient Height, and an Encounter ID which may include a Room, a Bed, and a Building (including CCA). The program pump request may also include Order Information or “order details”, including an Order ID, a Start Date/Time, a Stop Date/Time, a Route of Administration, a Rate, a Duration of Infusion (Infuse Over), a Total Volume to be Infused (VTBI), an Ad Hoc Order Indicator, and Ingredients including HIS Drug Name or HIS Generic Drug Name, HIS Drug Identifier or HIS Generic Drug ID, Rx Type (Additive or Base), Strength w/units, and Volume w/units. The program pump request may further include Patient Controlled Analgesia (PCA) Orders Only information, such a PCA Mode-PCA only, Continuous only, or PCA and Continuous, a Lockout Interval (in minutes), a PCA Continuous Rate, a PCA Dose, a Loading Dose, a Dose Limit, a Dose Limit Time w/units, a Total Volume in vial or syringe, and Order Comments.

The MMU server 3108 may map or convert the wide range of expressions of units allowed by the HIS 3110 or POC system 3125 for POC client 3126 requests into the much more limited set of units allowed in the MMU server 3108 and infusion pump 3130. For example, the POC client 3126 request may express “g, gm, gram, or grams” whereas the MMU server 3108 and/or infusion pump 3130 may accept “grams” only. Infusion pump 3130 delivery parameters or infusion pump 3130 settings are mapped or converted from corresponding order information or “order details” of the program pump request.

The MMU server 3108 may store in an associated memory a mapping or translation table that keep track of the logical ID, serial number or other identifier of an infusion pump 3130 and the corresponding current network (static or dynamic) address (Internet Protocol (IP) address) or ID of the infusion pump 3130 on the network, which in this example is a wireless network. The MMU server 3108 may be able to translate or associate a given identifier of the infusion pump 3130 with its network address in the translation table and provide the network IP address to the requesting POC system 3125 or device. The MMU server 3108 may also store in an associated memory and/or look up the drug library applicable to the scanned infusion pump 3130 and/or convert the Drug ID and Strength from the pump program request into an index number of the medication at the desired strength or concentration from the drug library. The duration of the infusion may come from the POC system 3125 in hours and minutes and may be converted to just minutes for the infusion pump 3130 to recognize it. Volume or VTBI may be rounded to provide a value-specific and infuser-specific number of digits to the right of the decimal point. Units (of drug) may be converted to million units where appropriate. Patient weight may be converted and either rounded according to infuser-specific rules or not sent to the infuser.

Once the MMU server 3108 transforms the information from the program pump request into infusion pump settings or delivery parameters and other information in a format acceptable to the infusion pump 3130, the MMU server 3108 may wireles sly download a command message to the infusion pump 3130. If the infusion pump 3130 is not already equipped with the latest appropriate version of the hospital-established drug library, the MMU server 3108 may also automatically download a drug library to the infusion pump 3130. The hospital-established drug library may be maintained in a separate process undertaken by the biomedical engineer or pharmacist 3122 to place limits on the programming of the infusion pump 3130, as well as other infusion pump operating parameters such as default alarm settings for air in the line, occlusion pressure, and the like. The drug library may set up acceptable ranges or hard and/or soft limits for various drug delivery parameters in the infusion pump 3130.

The MMU server 3108 may also download to the infusion pump new versions, patches, or software updates of the infusion pump's internal operating system software. The infusion settings or delivery parameters and other information from the MMU server 3108 may be entered into the memory of the infusion pump 3130 and the infusion pump 3130 settings may automatically populate the programming screen(s) of the infusion pump 3130, just as if the caregiver 3132 had entered the information and settings manually. The infusion pump 3130 screen may populate with the name of the drug and drug concentration based on the drug library index number, patient weight, rate, VTBI, and/or duration. Further, the MMU server 3108 may transmit one or more synchronization signals or screen content display rules/parameters to the infusion pump 3130. A return message of confirmation signal may be sent to the MMU server 3108 by the infusion pump 3130 to indicate that the command message has been received. At this point, if necessary, the caregiver 3104 may manually enter any additional infusion settings or optional information that was not included in the command message.

The infusion pump 3130 may then prompt the caregiver 3132 to start the infusion pump 3130 by pressing the start button. When the caregiver 3132 presses the start button, a confirmation screen with the infusion settings programmed may be presented for confirmation and an auto-program acknowledgment message can be sent to the MMU server 3108 to forward without request (e.g., pushed in a near real-time manner) or provide to the POC system 3125 when requested or polled. When the caregiver 3132 presses the button to confirm, the infusion pump 3130 may begin delivering fluid according to the programmed settings. The infusion pump 3130 may send a status message to the MMU server 3108 indicating that the infusion pump 3130 was successfully auto-programmed, confirmed and started by the caregiver 3132, and is now delivering fluid. This information may also be displayed at the infusion pump. The MMU server 3108 may continue to receive logs and status messages wirelessly from the infusion pump 3130 periodically as the infusion progresses or when alarms occur.

The MMU server 3108 may report a portion of the initial status message to the POC client 3126 through the POC server 3124 (in MMU format) to indicate that the infusion pump 3130 has been auto-programmed and the caregiver 3132 has confirmed the settings. The MMU server 3108 may communicate to the POC system 3125 and/or at the infusion pump 3130 the actual Rate, VTBI, and Duration. A notation at the bottom of the screen of the POC client and/or the infusion pump may indicate that the infusion pump 3130 is running. The infusion pump 3130 may compare and give a visual, audio, or other type of affirmative signal if the pump information matches or acceptably corresponds with the ordered information. An initial determination of whether the pump information matches the order may be done in the MMU server 3108 and communicated to the POC client 3126 through the POC server 3124. Alternatively, the POC server 3124 or the infusion pump 3130 may make the necessary comparisons. If the pump information does not match the order, the infusion pump 3130 at the display 88 may output a visual, audio, or other type of negative signal, which may include an error message.

The caregiver 3132 may be prompted to review and press a save button on the infusion pump 3130 if the order has been begun as desired or any variations are acceptable. The MMU server 3108 may receive status, event, differences, and variation information from the infusion pump 3130 and pass such information to the POC system 3125. In a separate subsequent step, the nurse may electronically sign the record and presses a send button on the POC client POC client 3126 to send the information to the patient's electronic medication record (EMR) or medication administration record (MAR).

### Additional Example Network Environment

FIG. 1B illustrates a network environment 100 in which a clinical environment 102 communicates with a cloud environment 106 via a network 104. The clinical environment 102 may include one or more healthcare facilities (e.g., hospitals). The network 104 may be any wired network, wireless network, or combination thereof. In addition, the network 104 may be a personal area network, local area network, wide area network, over-the-air broadcast network (e.g., for radio or television), cable network, satellite network, cellular telephone network, or combination thereof. For example, the network 104 may be a publicly accessible network of linked networks such as the Internet. For example, the clinical environment 102 and the cloud environment 106 may each be implemented on one or more wired and/or wireless private networks, and the network 104 may be a public network (e.g., the Internet) via which the clinical environment 102 and the cloud environment 106 communicate with each other. The cloud environment 106 may be a cloud-based platform configured to communicate with multiple clinical environments. The cloud environment 106 may include a collection of services, which are delivered via the network 104 as web services. The components of the cloud environment 106 are described in greater detail below with reference to FIG. 1C.

### Components of Clinical Environment

The clinical environment 102 may include one or more clinical IT systems, one or more infusion pumps, and optionally, one or more connectivity adapters. Further, the clinical environment 102 may be configured to provide cloud user interfaces (e.g., generated and provided by the cloud environment 106). The clinical IT system may include a hospital infusion system (HIS) designed to manage the facilities' operation, such as medical, administrative, financial, and legal issues and the corresponding processing of services. The HIS can include one or more electronic medical record (EMR) or electronic health record (EHR) systems, as well. The infusion pump is a medical device configured to deliver medication to a patient. The connectivity adapter is a network component configured to communicate with other components of the clinical environment 102 and also communicate with the cloud environment 106 on behalf of the other components of the clinical environment 102. In one embodiment, all messages communicated between the clinical environment 102 and the cloud environment 106 pass through the connectivity adapter. In some cases, the connectivity adapter is a network appliance with limited storage space (e.g., memory and/or persistent storage). The cloud user interfaces may be provided to a user in the clinical environment 102 via a browser application, desktop application, mobile application, and the like. The user may access status reports and other data stored in the cloud environment 106 via the cloud user interfaces.

The components of the clinical environment 102 may communicate with one or more of the other components in the clinical environment 102. For example, each of the clinical IT system and the infusion pump may communicate with the connectivity adapter via physical local area network (LAN) and/or virtual LAN (VLAN). The clinical environment 102 may include other medical devices and non-medical devices that facilitate the operation of the clinical environment 102.

### Components of Cloud Environment

FIG. 1C illustrates one embodiment of a cloud environment 102, which includes can include one or more of a drug library manager (DLM) 402, report manager 404, device manager 406, data flow manager (DFM) 408, cloud manager (CM) 410, data analyzer (DA) 412, and database 414.

The DLM 402 may provide a set of features and functions involved in the creation and management of drug libraries for use with infusion pumps. These drug libraries may provide user-defined settings for pump configuration and drug infusion error reduction.

The report manager 404 may provide various reporting capabilities for clinically relevant infusion data which users can choose to use for further analysis, such as tracking and trending of clinical practices.

The device manager 406 may oversee and manage the maintenance of infusion pumps, providing users the capability to view and manage asset and operational data. For example, the device manager 406 may schedule drug library and software updates for infusion pumps.

The DFM 408 may facilitate storing, caching, and routing of data between compatible infusion pumps 204, connectivity adapters 206, cloud services (e.g., infusion pump management software including modules 402-414 of FIG. 1C), and external systems. For example, the DFM may store infusion and operational data received from infusion pumps, store and cache infusion pump drug libraries and software images, convert and route network messaging between the cloud environment 106 and the clinical environment 102, convert and route medication order information from a hospital information system to an infusion pump (e.g., auto-programming or smart-pump programming), and/or convert and route alert information and infusion events from infusion pumps to hospital information systems (e.g., alarm/alert forwarding, and auto-documentation, or infusion documentation).

The CM 410 may serve as a general-purpose computing platform for the other modules illustrated in FIG. 1C. Functionally, the CM 410 may be similar to Microsoft Windows® or Linux® operating systems as it provides the following services: networking, computation, user administration and security, storage, and monitoring.

The DA 412 may provide data analytics tools for generating user interfaces and reports based on the data generated and/or received by the other modules illustrated in FIG. 1C.

The database 414 may store data generated and/or received by the modules 402-412 of the cloud environment 106. Although not illustrated in FIG. 1C, the cloud environment may provide other resources such as processors, memory, disk space, network, etc. The modules 402-412 may be hardware components configured to perform one or more of the techniques described herein. Alternatively, the modules 402-412 may be implemented using software instructions stored in physical storage and executed by one or more processors. Although illustrated as separate components, the modules 402-412 may be implemented as one or more hardware components (e.g., a single component, individual components, or any number of components), one or more software components (e.g., a single component, individual components, or any number of components), or any combination thereof.

In some embodiments, the cloud environment 106 can be implemented using a commercial cloud services provider (e.g., Amazon Web Services®, Microsoft Azure®, Google Cloud®, and the like). In other embodiments, the cloud environment can be implemented using network infrastructure managed by the provider and/or developer of the modules 402-412 shown in FIG. 1C. In some embodiments, the features and services provided by one of more of the modules 402-412 may be implemented on one or more hardware computing devices as web services consumable via one or more communication networks. In further embodiments, one of more of the modules 402-412 are provided by one or more virtual machines implemented in a hosted computing environment. The hosted computing environment may include one or more rapidly provisioned and released computing resources, such as computing devices, networking devices, and/or storage devices.

### Other Environments

FIGS. 1A-1C illustrate example environments in which the various techniques of the present disclosure may be utilized. However, the embodiments described herein are not limited to such environments and may be applied to any networked or non-networked environment. An example infusion pump that may be used in one or more of such environments is described below with reference to FIG. 2.

### Architecture of Infusion Pump

With reference to FIG. 2, the components of an example infusion pump are described in greater detail. The example architecture of the infusion pump 304 depicted in FIG. 2 includes an arrangement of computer hardware and software modules that may be used to implement aspects of the present disclosure. The infusion pump 304 may include many more (or fewer) elements and/or sub-elements than those shown in FIG. 2. It is not necessary, however, that all of these elements be shown in order to provide an enabling disclosure.

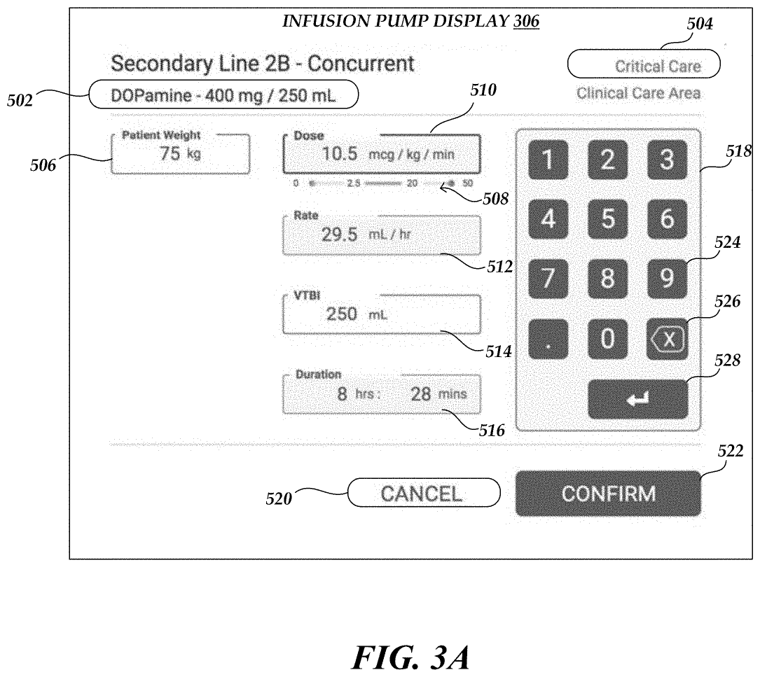

As illustrated, the infusion 304 includes a display 306, a processor 308, a network interface 310, and a memory 312, all of which may communicate with one another by way of a communication bus. The display 306 may display information generated or stored by the infusion pump 304 or any other information associated with the infusion pump 304. For example, infusion pump 304 may be used to deliver medication to a patient. In such a case, the display 306 may display the volume of the medication infused so far, the volume of the medication to be infused, the rate at which the medication is being infused, and the like. Examples of such displays are illustrated in FIG. 3A through FIG. 3D. The display 306 may also provide a keypad to the user for data entry and programming.

The processor 308 may receive information and instructions from other computing systems or services via a network. The processor 308 may also transmit information to and receive information from the memory 312 and further provide content to the display 306 for display. The network interface 310 may provide connectivity to one or more networks or computing systems in the network environment described herein. For example, the network interface 310 may be a serial port, a parallel port, or any other communication interface that can enable or facilitate wired or wireless communication according to any communication protocols such as Zigbee (e.g., IEEE 802.15.4), Bluetooth, Wi-Fi (e.g., IEEE 802.11), Near Field Communication (NFC), and the like.

The memory 312 may contain computer program instructions (grouped as modules in some embodiments) that the processor 308 can execute in order to implement one or more aspects of the present disclosure. The memory 312 may include RAM, ROM, and/or other persistent, auxiliary, or non-transitory computer-readable media. In some embodiments, the memory 312 stores an operating system that provides computer program instructions for use by the processor 308 in the general administration and operation of the infusion pump 304. As illustrated in FIG. 2, the memory 312 may include a patient weight check 314. In some embodiments, the patient weight check 314 implements various aspects of the present disclosure.

Although not shown in FIG. 2, the infusion pump 304 may further include one or more input devices such as a touch screen, mechanical buttons, or a voice recognition system. Further, the infusion pump 304 may include one or more additional storage devices for storing data generated by the infusion pump 304 or other data utilized in implementing aspects of the present disclosure.

### Infusion Pump Requiring Weight Entry Confirmation

With reference now to FIGS. 3A-3B, example infusion pump displays illustrating pump programming and operation of a patient weight check will be described. FIGS. 3A-3B illustrate embodiments of an infusion pump display 306. The display 306 includes various context parameters 502, 504, a graphical limit indicator 508, data entry fields 510, 512, 514, 516, a keypad 518, and various action buttons 520, 522. The context parameters include a drug selection field 502 and a clinical care area (“CCA”) selection field 504. The graphical limit indicator 508 indicates lower and upper hard limits (0 mcg/kg/min and 50 mcg/kg/min in the illustrated example) as well as lower and upper soft limits (2.5 mcg/kg/min and 20 mcg/kg/min in the illustrated example). The data entry (or treatment parameter) fields include a patient weight entry field 506, a drug dosing field 510, an infusion rate field 512, a volume-to-be-infused field 514, and an infusion duration field 516. The graphical limit indicator 508 may appear beneath a particular data entry field once the data entry field is selected. The numbers on the graphical limit indicator 508 will vary depending upon the selected context parameters and the selected data entry field. The keypad 518 includes various numerical keys 524 (digits 1-9 and a decimal point key), a backspace key 526 and an ENTER key 528. The numerical keys 524 are used by the user to input values into each of the data entry fields. The action buttons include a CANCEL key 520 and a CONFIRM key 522. The CANCEL key 520 may be used by the user to cancel all entries made, but not yet confirmed. The CONFIRM key 522 may be used by the user to confirm that all values entered are to be submitted to the infusion pump's microprocessor for further processing and to execute an infusion program according to the settings and values shown on the pump's display 306.

In the display 306 of FIG. 3A, the user has selected the drug DOPamine at a concentration of 400 mg/250 mL for infusion to the medical patient using the drug selection field. In one embodiment, when the drug selection field 502 is selected, a list of drugs at particular concentrations are presented in a drop-down menu. In the illustrated embodiment, the drug selection field 502 presents the user with the option to select drug and drug concentration via the same field (e.g., the dropdown menu may present “DOPamine—400 mg/250 mL” as one selectable value, and “DOPamine—200 mg/250 mL” as a second selectable value). In other embodiments (not illustrated) drug and drug concentration are independently selectable by the user via two distinct fields, such as a drug selection field 502 and a drug concentration selection field (not shown). For example, the drug “DOPamine” may be one of several drugs selectable by the user via the drug selection field 502, and the concentration “400 mg/250 mL” may be one of several drug concentrations selectable by the user via a drug concentration selection field (not shown). The methods described herein are applicable to both embodiments and references to using the drug selection as a context parameter 502 refers to using both a drug selection and drug concentration selection as context parameters. The user has also used the CCA selection field 504 to select the “Critical Care” CCA. Using one or more of these context parameters 502, 504 the infusion pump accesses a drug library and determines lower and upper soft and hard limits to be used by the pump to restrict further data entry and programming by the user. Since the user has selected the dose field 510 for setting the infusion therapy dose value, the lower and upper soft and hard limits are graphically displayed on a graphical limit indicator 508 positioned beneath the dose field 510. The graphical limit indicator 508 indicates that for the selected context parameters 502, 504, the lower and upper soft limits for the dosing parameter are 2.5 and 20 mcg/kg/min and the lower and upper hard limits for the dosing parameter are 0 and 50 mcg/kg/min, respectively. In the display 306 of FIG. 3A, the user has entered the value 10.5 mcg/kg/min, which between the lower and upper soft limits, and therefore a valid value that may be entered without restriction. In the illustrated embodiment, the user has also entered the patient's weight (in kilograms) in the patient weight field 506. The patient's weight is used as a treatment parameter in connection with the dose value (which may be entered in terms of mcg/kg/min or micrograms of drug per kilogram of patient weight per minute). In some embodiments, the dose value is entered in terms of mcg/min, or micrograms of drug per minute, independent of the patient's weight.

FIG. 3B illustrates the display 306 of the infusion pump after the patient weight check has determined that the entered patient weight value (the therapy patient weight) may have been incorrectly entered. For example, the patient weight check has determined that a difference between the value entered in the patient weight field 506 (75 kg in the illustrated embodiment) and a reference patient weight value exceeds a threshold condition. For example, the patient weight check may have determined that the entered patient weight value is 20% or more greater than the reference patient weight value. In response to such determination, an indicator 540 is displayed on the display 306. The indicator 540 requires the user to confirm the entered weight value before therapy may be initiated. The indicator 540 may display a message (as shown in FIG. 3B), as well as display a CONFIRM key (which may be incorporated into the message) for the user to activate to confirm the entered weight value. Alternatively, the user may be able to confirm the weight value by pressing the CONFIRM key 522.

### Method of Protecting Against Patient Weight Entry Errors

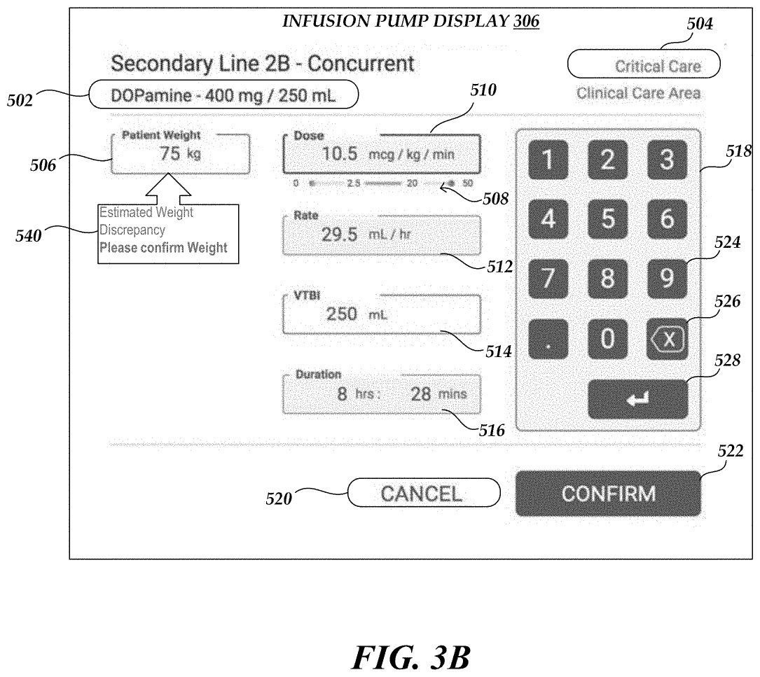

With reference now to FIG. 4, an example patient weight check method 700 will be described. The example method 700 may be performed, for example, by the infusion pump 304 of FIGS. 2-3B (or one or more components thereof, such as the patient weight check 314). The method 700 illustrates an example algorithm that may be programmed, using any suitable programming environment or language, to create machine code capable of execution by a CPU or microcontroller of the infusion pump 304. Various embodiments may be coded using assembly, C, OBJECTIVE-C, C++, JAVA, or other human-readable languages and then compiled, assembled, or otherwise transformed into machine code that can be loaded into read-only memory (ROM), erasable programmable read-only memory (EPROM), or other recordable memory of the infusion pump 304 that is coupled to the CPU or microcontroller and then then executed by the CPU or microcontroller. For convenience, the steps of the example method 700 are described as being performed by the patient weight check 314 of infusion pump 304.

The method 700 begins at block 702. At block 704 the patient weight check 314 determines a therapy patient weight value. For example, the patient weight check 314 may receive a value from a keypad that may be used by a user to manually enter the patient's weight. In another example, the patient weight check 314 may receive a therapy patient weight value from a memory location, such as from an EMR associated with the medical patient. The therapy patient weight value may be entered by a user via a display and keypad, including, but not limited to, any of the displays and keypads of FIGS. 3A-B.

At block 706 the patient weight check 314 determines one or more reference patient weight values. For example, the reference patient weight value may be received from or determined from data received from an external device configured to measure or estimate the patient's weight. One example of such device is a mid-arm circumference weight estimator, as illustrated and described below with respect to FIGS. 5-6B. The reference patient weight value may be received via wired or wireless communication with the external device. The therapy weight value and the reference patient weight value(s) are stored in a memory and compared at block 708. At block 710, the patient weight check 314 determines if a threshold error has occurred. For example, the patient weight check 314 determines if the difference between the therapy and reference patient weight values exceeds a threshold amount. The threshold amount can correspond to a predetermined percentage of either the therapy or reference patient weight values. For example, if the difference is more than 5%, 10%, 20%, or 30% of the therapy patient weight value, a threshold error is determined to have occurred.

If a threshold error has occurred, the method proceeds to block 712 where an indication of the error is presented to the user. The user may also be prompted to provide a confirmation that the entered therapy patient weight value is correct. Once the user provides the requested confirmation (e.g., by keypress on a keyboard or keypad of the infusion pump display, etc.), the method proceeds to block 714. In addition, if at block 714 the patient weight check 314 determines that a threshold error has not occurred, the method proceeds directly to block 714.

At block 714, the entered therapy patient weight value is used to determine other treatment parameters, such as infusion rate, volume-to-be-infused, and/or infusion duration of time. The infusion pump initiates and provides an infusion therapy using the determined treatment parameters at block 714, as well. The method 700 proceeds to block 716 and ends.

### External Device for Weight Estimation

FIG. 5 illustrates one embodiment of an external device 800 that may be used to estimate a patient's weight. A device used to estimate a patient's weight may sometimes be referred to as a weight measurement device. A weight measurement device may either measure or estimate a weight, or an indicator of weight (e.g., mass, body surface area, volume, etc.). The external device 800 of FIG. 5 is just one example of a device that can provide a weight measurement, or an estimate of the patient's weight. Other devices may be used as well. For example, in some embodiments, the external device is a scale, an imaging device, a laser scanning device, or a combination. For example, such devices may use imaging and/or 3D laser-based anthropometry or scanning to measure body surface area. In some cases, body surface area is used instead of a weight estimate. In some cases, a body surface area measurement is used to estimate the patient's weight. The external device 800 of FIG. 5 is a mid-arm circumference (“MAC”) weight estimator. The external device 800 measures the circumference and the length of the patient's arm (from elbow to mid-arm between elbow and shoulder). The device 800 may estimate the patient's weight from these linear measurements, or it may provide the linear measurements to an infusion pump. In such case, the infusion pump may be configured to estimate the patient's weight from the linear measurements of the external device 800.

The external device 800 includes a housing 802, from which three flexible tapes 804, 806, 808 extend and retract. The housing 802 is positioned mid-arm of the patient (between elbow and shoulder). The upper and lower vertical tapes 804, 806 are extended until they reach the patient's shoulder and elbow, respectively. The horizontal tape 808 is extended from the housing 802 and wrapped around the mid-arm of the medical patient in the direction of arrows 812, 814. A button 810 located on the horizontal tape 808 is positioned over a corresponding button 816 positioned on the housing 802 to complete the measurement of the circumference of the patient's arm. The buttons 810, 816 may be magnetic.

The linear measurements from the external device 800 may be referred to as MAC data. The MAC data (or an estimate of the patient's weight determined by the external device 800 from the MAC data) may be communicated from the external device 800 via a communication link 818. The communication link 818 may include either a wired (as shown) communication link, or a wireless communication link. The wireless communication link can include a BLUETOOTH, near-field communication or other wireless communication link. A correlation, or a linear equation describing the correlation, between MAC data of a general population and weight data of the general population may be used by the external device 800 or an infusion pump, to estimate the particular patient's weight from the particular patient's MAC data.

FIGS. 6A-C illustrate various embodiments of tape assemblies 820, 832, 840, 850 that may be used alone or together as part of an external device 800 for weight estimation. In the embodiment of FIG. 6A, upper and lower tape assemblies 820, 832 are coupled to each other, and are used to measure the length of a patient's arm. The upper assembly 820 includes the upper tape 804, a gear housing 824, and a toothed gear 828. The lower assembly 832 includes the lower tape 806, a gear housing 826, and a toothed gear 830. The gears 828, 830 may be coupled to each other. A detector (or detectors) (not shown) such as a photodetector, LED-phototransistor pair, Hall effect sensor, or other sensor is used to determine the number of rotations of the gears 828, 830 as the upper and lower tapes 804, 806 are extended from their respective tape housings 820, 832. The length of the extended tape 822 corresponds to the number of rotations of the gears. Therefore, such detector can be used to determine the length of the medical patient's arm.

FIG. 6B illustrates an embodiment of a tape assembly 840 that includes only a single housing 842, gear 844, and detector (not shown). The upper and lower tapes 804, 806 are incorporated into the single housing 842. FIG. 6C illustrates an embodiment of a horizontal tape assembly 850. The horizontal tape assembly includes a horizontal tape 808, housing 852, gear 854, and detector (not shown). The horizontal tape assembly may be used to measure the circumference of the patient's arm 856, as described above.

### Other Considerations

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

Many other variations than those described herein will be apparent from this disclosure. For example, depending on the embodiment, certain acts, events, or functions of any of the algorithms described herein can be performed in a different sequence, can be added, merged, or left out altogether (e.g., not all described acts or events are necessary for the practice of the algorithms). Moreover, in certain embodiments, acts or events can be performed concurrently, e.g., through multi-threaded processing, interrupt processing, or multiple processors or processor cores or on other parallel architectures, rather than sequentially. In addition, different tasks or processes can be performed by different machines and/or computing systems that can function together.

The various illustrative logical blocks, modules, and algorithm elements described in connection with the embodiments disclosed herein can be implemented as electronic hardware, computer software, or combinations of both. To clearly illustrate this interchangeability of hardware and software, various illustrative components, blocks, modules, and elements have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described in connection with the embodiments disclosed herein can be implemented or performed by a machine, such as a processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, or algorithm described in connection with the embodiments disclosed herein can be embodied directly in hardware, in a software module stored in one or more memory devices and executed by one or more processors, or in a combination of the two. A software module can reside in RAM memory, flash memory, ROM memory, EPROM memory, EEPROM memory, registers, hard disk, a removable disk, a CD-ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC. The ASIC can reside in a user terminal. In the alternative, the processor and the storage medium can reside as discrete components in a user terminal.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements, and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Unless otherwise explicitly stated, articles such as “a”, “an”, or “the” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B, and C” can include a first processor configured to carry out recitation A working in conjunction with a second processor configured to carry out recitations B and C.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, certain embodiments described herein can be implemented within a form that does not provide all of the features and benefits set forth herein, as some features can be used or practiced separately from others. All such modifications and variations are intended to be included herein within the scope of this disclosure. Further, additional embodiments created by combining any two or more features or techniques of one or more embodiments described herein are also intended to be included herein within the scope of this disclosure.

What is claimed is:

1. An infusion pump configured to determine a confidence in a patient weight value used to determine one or more treatment parameters, the infusion pump comprising:
a display configured to display a keypad and to receive input from a user via the keypad;
a processor in communication with the display; and
a memory in communication with the processor and configured to store instructions that when executed by the processor cause the execution of a patient weight check configured to:
determine a patient weight value corresponding to a weight of a medical patient to receive an infusion therapy from the infusion pump according to one or more treatment parameters;
determine a reference weight value corresponding to a weight estimate of the medical patient;
determine that a difference between the patient weight value and the reference weight value violates a threshold condition;
provide an indication to the user that the difference between the patient weight value and the reference weight value violates the threshold condition;
receive a confirmation associated with the patient weight value;
determine the one or more treatment parameters using the patient weight value; and
provide the infusion therapy to the patient according to the one or more treatment parameters.

2. The infusion pump of claim 1, wherein the patient weight check is configured to determine the patient weight value from the input received from the user via the keypad.

3. The infusion pump of claim 1, wherein the patient weight check is configured to determine the patient weight value from data received over a network.

4. The infusion pump of claim 3, wherein the data is received from an electronic medical record.

5. The infusion pump of claim 1, wherein the patient weight check is configured to receive the patient weight value from a first source and receive the reference value from a second source different than the first source.

6. The infusion pump of claim 1, wherein the reference weight value is determined from data received from a patient weight measurement device.

7. The infusion pump of claim 6, wherein the patient weight measurement device comprises a scale, a device that estimates a weight from an image of at least a portion of the patient, or a laser scanning device configured to scan at least a portion of the patient.

8. The infusion pump of claim 6, wherein the patient weight measurement device comprises a mid-arm circumference measurement accessory.

9. The infusion pump of claim 1, wherein the patient weight check is configured to determine that the difference between the patient weight value and the reference violates a threshold condition by determining that the reference weight value is at least 20% different than the patient weight value.

10. The infusion pump of claim 1, wherein the threshold condition may be configured by an operator.

11. The infusion pump of claim 1, wherein the indication to the user that the difference between the patient weight value and the reference weight value violates the threshold condition comprises a message for presentation on the display.

12. The infusion pump of claim 1, wherein the one or more treatment parameters comprises one or more of a dose, an infusion rate, a volume to be infused, an infusion duration, or a patient weight.

13. A method of determining confidence in a patient weight value used to determine one or more treatment parameters, the method comprising:
determining a patient weight value corresponding to a weight of a medical patient to receive therapy from a medical device according to one or more treatment parameters;
determining a reference weight value corresponding to a weight estimate of the medical patient;
determining that a difference between the patient weight value and the reference weight value violates a threshold condition;
providing an indication to the user that the difference between the patient weight value and the reference weight value violates the threshold condition;
determining the one or more treatment parameters using the patient weight value; and
providing the infusion therapy to the patient according to the one or more treatment parameters.

14. The method of claim 13, further comprising receiving a confirmation associated with the patient weight value.

15. The method of claim 13, wherein determining the patient weight value comprises determining the patient weight value from an input received at a keypad of the medical device.

16. The method of claim 13, wherein determining the patient weight value comprises determining the patient weight value from data received over a network.

17. The method of claim 15, determining the patient weight value comprises determining the patient weight value from data received from an electronic medical record.

18. The method of claim 13, wherein determining the patient weight value comprises receiving the patient weight value from a first source and wherein determining the reference weight value comprises receiving the reference value from a second source different than the first source.

19. The method of claim 13, determining the reference weight value comprises receiving data from a patient weight measurement device and determining the reference weight value from the data received from the patient weight measurement device.

20. The method of claim 18, wherein the patient weight measurement device comprises a scale, a device that estimates a weight from an image of at least a portion of the patient, or a laser scanning device configured to scan at least a portion of the patient.

21. The method of claim 18, wherein the patient weight measurement device comprises a mid-arm circumference measurement accessory.

22. The method of claim 13, wherein determining that the difference between the patient weight value and the reference violates a threshold condition comprises determining that the reference weight value is at least 20% different than the patient weight value.

23. The method of claim 13, wherein the threshold condition may be configured by an operator.

24. The method of claim 13, wherein providing the indication to the user that the difference between the patient weight value and the reference weight value violates the threshold condition comprises displaying a message on a display of the medical device.

25. The method of claim 13, wherein the medical device comprises an infusion pump.

26. The method of claim 25, wherein the one or more treatment parameters comprises one or more of a dose, an infusion rate, a volume to be infused, an infusion duration, or a patient weight.
